(19) United States
(12) Patent Application Publication
(10) Pub. No.: US 2024/0285949 A1
(43) Pub. Date: Aug. 29, 2024

(54) MODULATION OF SESTRINS

(71) Applicant: Leonhardt Ventures LLC, Corona Del Mar, CA (US)

(72) Inventors: Howard J. Leonhardt, Mission Viejo, CA (US); Jorge Genovese, Irvine, CA (US)

(21) Appl. No.: 18/587,673

(22) Filed: Feb. 26, 2024

Related U.S. Application Data
(60) Provisional application No. 63/487,516, filed on Feb. 28, 2023.

(51) Int. Cl. A61N 1/36

(52) U.S. Cl. CPC A61N 1/36121; A61N 1/36034; A61N 1/36157; A61N 1/36171; A61N 1/36175

(57) ABSTRACT

Described is a low voltage, pulsed electrical stimulation device for modulating the expression of sestrin, a useful protein, by tissues. Also described are methods of enhancing expression of sestrins in cells, particularly a method of stimulating the expression and/or release of sestrins in a cell having a gene encoding a sestrin, wherein the method includes applying a bioelectric signal comprising a biphasic constant current at a frequency of, within 15%, 10 Hz to 20 Hz, with a pulse width of, within 15%, 300 to 400 microseconds (μsec).

### CROSS-REFERENCE TO RELATED APPLICATION

This application claims the benefit under 35 U.S.C. § 119(e) of U.S. Provisional Patent Application Ser. No. 63/487,516, filed Feb. 28, 2023, the disclosure of which is hereby incorporated herein in its entirety by this reference.

### TECHNICAL FIELD

The application relates generally to the field of medical devices and associated methods of treatment, and more specifically to methods of treatment involving the precise bioelectrical stimulation of a subject's tissue. More specifically, the application relates to a device, programmed to produce bioelectric signals, and associated methods for the controlled expression of secreted sestrins via application of such bioelectric signals.

### BACKGROUND

Sestrins (Sesns) are a family of highly conserved, stress-responsive proteins. Sestrins have been identified in numerous studies to have powerful anti-aging properties. Studies have demonstrated that increased circulating sestrins can help increase the benefits of exercise in less exercise time.

Sestrins are known to protect organisms against various noxious stimuli including DNA damage, oxidative stress, starvation, endoplasmic reticulum stress, and hypoxia. Sestrins regulate metabolism mainly through activation of the key energy sensor AMP-dependent protein kinase (AMPK) and inhibition of mammalian target of rapamycin complex 1 (mTORC1). Sestrins also play pivotal roles in autophagy activation and apoptosis inhibition in normal cells, while conversely promoting apoptosis in cancer cells. The functions of Sestrins in diseases such as metabolic disorders, neurodegenerative diseases, cardiovascular diseases, and cancer have been broadly investigated in the past decades.

It would be an improvement in the art to have a way to modulate the expression of sestrins.

### BRIEF SUMMARY

Described herein is a bioelectric stimulator particularly configured to modulate (e.g., upregulate or downregulate) the expression and/or release of sestrins in cellular tissue.

Further described is a precise bioelectric signal that upregulate or downregulate levels of circulating sestrins on demand with control.

Particularly described is a bioelectric stimulator comprising an electric signal generator and electrode(s), which electric signal generator is programmed to produce at least one bioelectric signal that stimulates target tissue comprising living cells so as to modulate expression and/or release of sestrins by the living cells of the target tissue. In certain embodiments, the bioelectric signal comprises, within 15%, a biphasic constant current at a frequency of, within 15%, 10 Hz to 20 Hz, with a pulse width of, within 15%, 300 to 400 microseconds (μsec).

Also described are methods of using such a bioelectric stimulator to stimulate target tissue comprising living cells of a subject to modulate the expression of sestrins by the living cells, the method comprising: administering the bioelectric signal to the living cells via the electrode(s) for from about 5 minutes to about an hour, so as to modulate the expression of sestrins by the living cells.

Sestrins have antiaging activity and are considered to prevent age-associated pathologies prevention. They also have antiaging activity associated with antioxidant effects. They have been suggested for cartilage homeostasis and protection and treating and preventing osteoarthritis, intervertebral disc protection, treating or preventing diabetes, cardiovascular protection (including atrial fibrillation), liver protection, exercise benefits and muscle health, neuro, strokes, antitumor activity, immunology, and stress control.

In certain embodiments, the described methods may be used to treat a subject suffering from heart disease, cancer, muscle atrophy, skin disorder(s), hair loss, Alzheimer's disease, Parkinson's disease, dementia, cognitive function, pre-mature aging related disorder, senescence, aging, muscle atrophy, and/or inflammation.

Also described is a method of inhibiting expression of sestrins in a subject utilizing a bioelectric stimulator, wherein the bioelectric stimulator comprises an electric signal generator and associated electrode(s), which electric signal generator is programmed to produce at least one bioelectric signal that stimulates target tissue comprising living cells so as to inhibit expression and/or release of sestrins by the living cells of the target tissue.

### BRIEF DESCRIPTION OF THE DRAWINGS

FIG. 1 depicts a programmed bioelectric stimulator for delivery to a subject connected to multiple soft conductive electrode pads.

FIG. 2 depicts a programmed bioelectric stimulator as described herein.

FIG. 3 depicts a conductive soft wrap which may be used with the described system.

FIG. 4 depicts a programmed bioelectric stimulator depicted alongside a pen.

FIG. 5 depicts a bioelectric stimulation system for laboratory testing.

### DETAILED DESCRIPTION

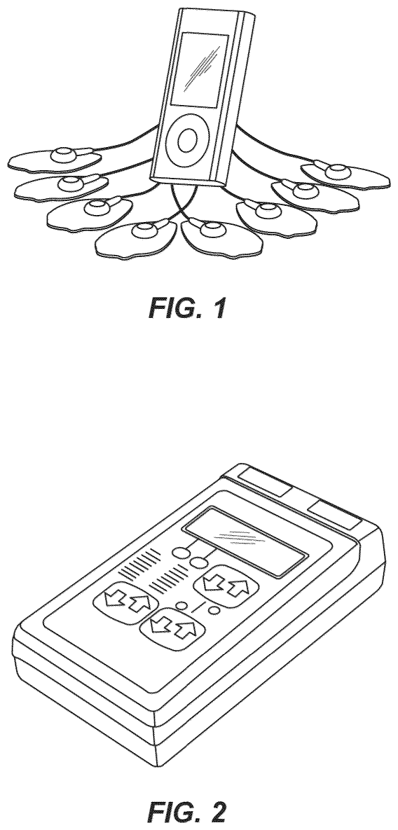

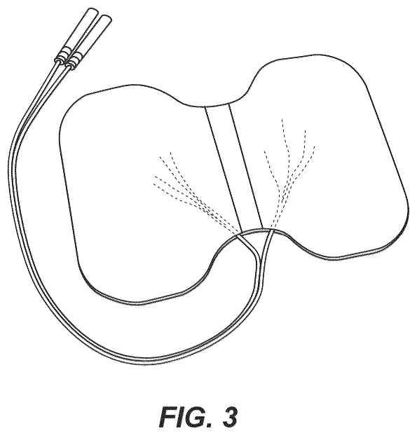

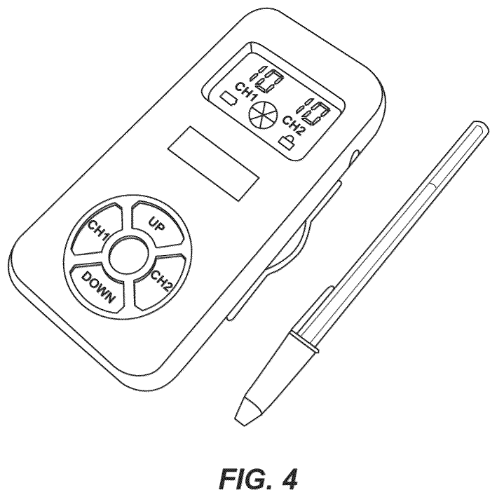

Referring now to FIG. 1, depicted is a stimulator for use in treating a human. The depicted device is about the size of a pen (FIG. 4) and is programmable.

A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, California, US or HTM Electrônica of Amparo, BR. The leading pacemaker manufacturers are Medtronic, Boston Scientific Guidant, Abbott St. Jude, BioTronik and Sorin Biomedica.

Construction of the electric signal generators and pacemakers are known in the art and can be obtained from OEM suppliers as well as their accompanying chargers and programmers. The electric signal generators are programmed to produce specific bioelectric signals to lead to specific protein expressions at precisely the right time for, e.g., optimal treatment or for tissue regeneration.

The biostimulator of FIG. 1 is depicted with multiple soft conductive electrode pads. Electrodes may be used to deliver a bioelectric signal to the subject by applying the electrodes to the subject's skin (e.g., on the skin above the thigh muscles or on the skin above the kidneys). In certain embodiments, a bioelectric stimulator is in electrical connection with a conductive soft wrap.

A bench top stimulator (e.g., a Mettler Model 240 Stimulator from Mettler Electronics of Anaheim, CA, US) may be pre-programmed with the bioelectric signaling sequence(s) for controlling the expression and/or release of SESTRINS.

Described herein is the repeated reliability of increasing selected sestrin protein family expressions by up to 190% with only 30 minutes of stimulation time across a variety of tissue and cell types including muscle derived myoblasts.

In some embodiments, the application of bioelectric signals can further be used to modulate (e.g., upregulate) expression by the subject's cells the production of other biological molecules in addition to sestrins. Sec, e.g., U.S. Pat. No. 10,960,206 to Leonhardt et al. (Mar. 30, 2021) for “Bioelectric Stimulator,” the contents of which are incorporated herein by this reference.

For example, in certain embodiments (e.g., in order to provide antiaging effects, cognitive improvements, sexual health, and/or cardiovascular protection), further specific bioelectric signals are applied that upregulate expression of Nanog, klotho, and sirtuin.

Further, in certain embodiments, bioelectric controlled modulating (e.g., upregulating) expression of the sestrin family of proteins is added to bioelectric signals for modulating expression and/or release of klotho, follistatin, tropoclastin, stromal cell-derived factor-1 (“SDF1”) (a stem cell recruiting signal), platelet-derived growth factor (“PDGF”), and/or insulin-like growth factor 1 (“IGF-1”) for use, for example, in anti-aging therapy and muscle atrophy recovery. Sec, e.g., the incorporated U.S. Pat. No. 10,960,206 to Leonhardt et al. for such bioelectric signals.

The treatment and/or prevention of depression may involve the application of further bioelectric signal(s) to upregulate expression of Klotho. See, e.g., U.S. Patent Application Publication US 2020-0289826-A1 to Leonhardt et al. (Sep. 17, 2020) for “Klotho Modulation” and U.S. Patent Application Publication US 20210402184-A1 to Leonhardt et al. (Dec. 31, 2021) for “Klotho Modulation.”

An implantable medical lead is described in U.S. Pat. No. 8,442,653 to Gill (May 14, 2013) for “Brain Electrode,” the contents of which are incorporated herein by this reference.

Both wireless non-invasive and/or implantable wire lead (“electrode”) based means may be used to deliver the regeneration and healing promoting bioelectric signal(s) to target organs such as the brain.

A wireless, single lumen infusion pacing lead or infusion conduction wide array patch may all be used to deliver the regeneration signals and substances to the organ of interest to be treated or they may be used in combination.

A re-charging wand for use herein is preferably similar to the pacemaker re-charging wand developed by Alfred Mann in the early 1970s for recharging externally implantable pacemakers.

Bioelectric stimulation can be done with the described bioelectric stimulator, which can have a pacing infusion lead with, e.g., a corkscrew lead placed/attached at, e.g., the center of the tissue to be stimulated and/or treated.

The bioelectric stimulator is actuated and runs through programmed signals to signal the release of, e.g., sestrins. In such a method, the electrical signal may be measured three (3) mm deep into the tissue.

### Relationship Between the Components:

The micro voltage signal generator is attached to the pacing infusion lead with, e.g., a brain electrode (Medtronic) (e.g., for bioelectric stimulation of the brain), or conductive polymer bandage or patch to the tissue or organ to be treated. An external signal programmer may be used to program the micro voltage signal generator with the proper signals for treatment including the sestrins producing signal(s). The device battery may be re-chargeable with an external battery charging wand.

The essential elements are the micro voltage signal generator and the means for delivering the signal to the target tissue.

The signal generator may be external or internal. The transmission of the signal may be wireless, via liquid and/or via wires.

The tissue contact interface may be, e.g., a patch or bandage or may be via electrodes or leads. FDA cleared gel tape electrodes (Mettler) may be used for skin delivery. Electro acupuncture needles may be used to ensure the signals positively reach target tissues under the skin.

In certain preferred embodiments a method of stimulating the expression of sestrins in a living cell having a gene encoding a sestrins is described, wherein the method comprises: applying to the cell a bioelectric signal of, within 15%, 10 Hz to 20 Hz at, within 15%, 300 to 400 μsec pulse width duration, wherein the amount of sestrins expression enhanced by this bioelectric signal is greater than that seen with a generic bioelectric cell stimulation alone as may be determined by an analysis of the upregulation of mRNA level/GAPDH fold gene expression in the cell in each situation.

Sestrins activity in treating disease states and conditions is many fold. Sestrins have been identified in numerous studies to have powerful anti-aging properties. Studies have demonstrated that increased circulating sestrins can help increase the benefits of exercise in less exercise time. Sestrins may prevent atrophy of muscles from disuse or aging. They exhibit cardioprotective actions to help prevent hearts from damage. Sestrins seem to have a strong role in preventing or reversing aging related skin wrinkles. They could have a role in preventing hair loss. Via counter acting oxidative stress they may have a significant role in staving off neurodegenerative disorders such as memory-loss, Alzheimer's and Parkinson's disease. Sestrins possibly could have an important role in preventing cancer tumor growth progression.

There is increasing evidence to show that sestrin2 has pro-survival properties. Sestrin2 plays an important neuroprotective role after hypoxic-ischemic encephalopathy. Administration of rh-sestrin2 not only reduced infarct area and brain atrophy, but also significantly improved neurological function. An increase in sestrin2 expression as a result of brain-derived neurotrophic factor administration confers neuronal resistance against oxidative stress in primary rat cortical cultures. Sestrin2 induced by accumulation of amyloid b-peptide plays a protective role against amyloid neurotoxicity in primary cortical neurons possibly through the autophagy signaling pathway. Shi X, et al. Sestrin2, as a negative feedback regulator of mTOR, provides neuroprotection by activation AMPK phosphorylation in neonatal hypoxic-ischemic encephalopathy in rat pups. J Cereb Blood Flow Metab. 2017 April; 37(4): 1447-1460.

A growing body of evidence suggests that sestrins, especially sestrin2, can counteract oxidative stress, lessen mammalian/mechanistic target of rapamycin (mTOR) expression, and promote cell survival, thereby playing a critical role in aging-related disorders including neurodegeneration. Strategies capable of augmenting sestrin expression may carry clinical significance in neurodegenerative diseases. Chen S D, et al. Emerging Roles of Sestrins in Neurodegenerative Diseases: Counteracting Oxidative Stress and Beyond. J Clin Med. 2019 Jul. 9; 8(7): 1001.

The invention is further described by the following illustrative Examples.

### EXAMPLE(S)

### Example I

### Modulation of Expression of Sestrin1 (SESN1)

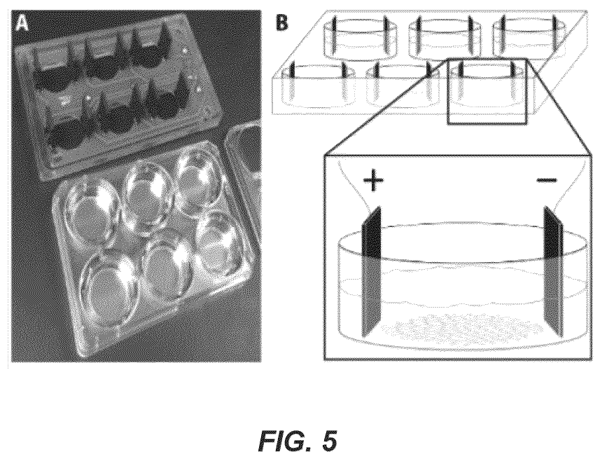

FIG. 5 depicts a bioelectric stimulation system. Cells and/or tissue are plated in each dish and cultured. Stimulation occurs using an electrode array (shown at the top of panel A), which is inverted and introduced into the 6-well dish where cells are grown. Each well receives uniform stimulation via a pair of carbon electrodes.

### Upregulation of Sestrin 1 (SESN1)

A biphasic constant current on skeletal muscle myoblasts of:

3 mA, 10 Hz (as expressed by a Mettler electric signal generator), 300 us for 30 minutes upregulated expression of SESN1 by 93.8% over control.

3 mA, 10 Hz, 300 us for 1 hour upregulated expression of SESN1 by 26% over control.

5 mA, 20 Hz, 400 us for 1 hour on dermal fibroblasts stimulated upregulated expression of SESN1 51% over control.

Downregulation of SESN1-longer treatment times have reduced treatment effectivity or induced downregulation (−9.5% for SESN1 after 90 min. treatment).

### Example II

### Modulation of Expression of Sestrin 2 (SESN2)

FIG. 5 depicts a bioelectric stimulation system. Cells and/or tissue are plated in each dish and cultured. Stimulation occurs using an electrode array (shown at the top of panel A), which is inverted and introduced into the 6-well dish where cells are grown. Each well receives uniform stimulation via a pair of carbon electrodes.

### Upregulation of Sestrin 2 (SESN2)

The following biphasic constant currents were applied to skeletal muscle myoblasts:

3 mA, 10 Hz (as expressed by a Mettler electric signal generator), 300 us for 30 min. upregulated expression of SESN2 190% over control.

3 mA, 10 Hz, 300 us for 1 hour upregulated expression of SESN2 162% over control.

A previous signal (5 mA, 20 Hz, 400 us for 1 hour were applied to dermal fibroblasts), which upregulated expression of SESN2 by 121% over control.

Using comparative quantitative PCR, a treatment of C2C12 (mouse myoblasts) at an intensity of 1 mA for 1 hour, measured an increase of 128% in SESN-1 expression compared to that of the untreated control.

Downregulation of SESN2-longer treatments have reduced treatment effectivity or induced downregulation (−48.4% for SESN2 after 90 min. treatment).

### REFERENCES

(The contents of the entirety of each of which is incorporated herein by this reference.)
Bae et al. “Sestrins activate Nrf2 by promoting p62-dependent autophagic degradation of Keap1 and prevent oxidative liver damage” Cell Metab. 2013 Jan. 8; 17(1):73-84. doi: 10.1016/j.cmet.2012.12.002. Epub 2012 Dec. 27. PMID: 23274085.
Chen et al. “Emerging Roles of Sestrins in Neurodegenerative Diseases: Counteracting Oxidative Stress and Beyond” J Clin Med. 2019 Jul. 9; 8(7): 1001. doi: 10.3390/jcm8071001. PMID: 31324048; PMCID: PMC6678886.
Chen et al. “Potential Roles of Sestrin2 in Alzheimer's Disease: Antioxidation, Autophagy Promotion, and Beyond” Biomedicines 2021 Sep. 24; 9(10): 1308. doi: 10.3390/biomedicines9101308. PMID: 34680426; PMCID: PMC8533411.
Ding et al. “p53-inducible SESTRINs might play opposite roles in the regulation of early and late stages of lung carcinogenesis” Oncotarget. 2019 Dec. 10; 10(65):6997-7009. doi: 10.18632/oncotarget.27367. PMID: 31857853; PMCID: PMC6916756.
Dong XC. “The potential of sestrins as therapeutic targets for diabetes” Expert Opin Ther Targets. 2015; 19(8): 1011-5. doi: 10.1517/14728222.2015.1044976. Epub 2015 May 5. PMID: 25944222; PMCID: PMC4504765.
Dong et al. “Upregulation of sestrins protect atriums against oxidative damage and fibrosis in human and experimental atrial fibrillation” Sci Rep. 2017 Apr. 11; 7:46307. doi: 10.1038/srep46307. PMID: 28397812; PMCID: PMC5387733.
Haidurov et al. “Sestrin family—the stem controlling healthy ageing,” Mech Ageing Dev. 2020 December; 192:111379. doi: 10.1016/j.mad.2020.111379. Epub 2020 Oct. 3. PMID: 33022334.
Kim et al. “Sestrin2 protects against acetaminophen-induced liver injury” Chem Biol Interact. 2017 May 1; 269:50-58. doi: 10.1016/j.cbi.2017.02.002. Epub 2017 Feb. 13. PMID: 28209544.
Kim et al. “Sestrins are evolutionarily conserved mediators of exercise benefits” Nat Commun. 2020 Jan. 13; 11(1):190. doi: 10.1038/s41467-019-13442-5. PMID: 31929512; PMCID: PMC6955242.
Kim et al. “Sestrins in Physiological Stress Responses” Annu Rev Physiol. 2021 Feb. 10; 83:381-403. doi: 10.1146/annurev-physiol-031620-092317. Epub 2020 Oct. 28. PMID: 33113341; PMCID: PMC7878365.
Lee et al. “Sestrin as a feedback inhibitor of TOR that prevents age-related pathologies,” Science 2010 Mar. 5; 327(5970):1223-8. doi: 10.1126/science.1182228. PMID: 20203043; PMCID: PMC2866632.
Lee et al. “Sestrins at the crossroad between stress and aging” Aging (Albany NY). 2010 June; 2(6):369-74. doi: 10.18632/aging.100157. PMID: 20606249; PMCID: PMC2919257.
Lee et al. “Sestrins orchestrate cellular metabolism to attenuate aging,” Cell Metab. 2013 Dec. 3; 18(6):792-801. doi: 10.1016/j.cmet.2013.08.018. Epub 2013 Sep. 19. PMID: 24055102; PMCID: PMC3858445.
Lenhare et al. “Physical exercise increases Sestrin 2 protein levels and induces autophagy in the skeletal muscle of old mice” Exp Gerontol. 2017 Oct. 15; 97:17-21. doi: 10.1016/j.exger.2017.07.009. Epub 2017 Jul. 17. PMID: 28729213.
Li et al. “Sestrin 2 Deficiency Exacerbates Noise-Induced Cochlear Injury Through Inhibiting ULK1/Parkin-Mediated Mitophagy” Antioxid Redox Signal. 2023 January; 38(1-3): 115-136. doi: 10.1089/ars.2021.0283. Epub 2022 Oct. 20. PMID: 35708118; PMCID: PMC9885551.
Liu et al. “AMPK binds to Sestrins and mediates the effect of exercise to increase insulin-sensitivity through autophagy” Metabolism. 2015 June; 64(6):658-65. doi: 10.1016/j.metabol.2015.01.015. Epub 2015 Jan. 31. PMID: 25672217.
Liu et al. “Sestrin 2 controls the cardiovascular aging process via an integrated network of signaling pathways” Ageing Res Rev. 2020 September; 62:101096. doi: 10.1016/j.arr.2020.101096. Epub 2020 Jun. 13. PMID: 32544433.
Liu et al. “Sestrin 2, a potential star of antioxidant stress in cardiovascular diseases” Free Radic Med. Biol 2021 Feb. 1; 163:56-68. doi: 10.1016/j.freeradbiomed.2020.11.015. Epub 2020 Dec. 10. PMID: 33310138. Martyn J A J and Kaneki M. “Muscle Atrophy and the Sestrins” N Engl J Med. 2020 Sep. 24; 383(13):1279-1282. doi: 10.1056/NEJMcibr2003528. PMID: 32966727.
Oliveira et al. “The potential role of sestrin 2 in liver regeneration” Free Radic Biol Med. 2021 Feb. 1; 163:255-267. doi: 10.1016/j.freeradbiomed.2020.12.027. Epub 2020 Dec. 28. PMID: 33359262.
Pereira et al. “Sestrins induce natural killer function in senescent-like CD8+ T cells” Nat Immunol. 2020 June; 21(6):684-694. doi: 10.1038/s41590-020-0643-3. Epub 2020 Mar. 30. PMID: 32231301.
Rai et al. “Exploration of Novel Anti-Oxidant Protein Sestrin in Frailty Syndrome in Elderly,” Aging Dis. 2018 Apr. 1; 9(2):220-227. doi: 10.14336/AD.2017.0423. PMID: 29896412; PMCID: PMC5963344.
Rai N and Dey S. “Protective response of Sestrin under stressful conditions in aging,” Ageing Res Rev. 2020 December; 64:101186. doi: 10.1016/j.arr.2020.101186. Epub 2020 Sep. 26. PMID: 32992045.
Ro et al. “Tumor suppressive role of sestrin2 during colitis and colon carcinogenesis” Elife. 2016 Feb. 25; 5:e12204. doi: 10.7554/eLife.12204. PMID: 26913956; PMCID: PMC4805551.
Ro et al. “SESTRINs: Emerging Dynamic Stress-Sensors in Metabolic and Environmental Health” Front Cell Dev Biol. 2020 Dec. 3; 8:603421. doi: 10.3389/fcell.2020.603421. PMID: 33425907; PMCID: PMC7794007.
Sánchez-Alvarez et al. “Sestrins as a Therapeutic Bridge between ROS and Autophagy in Cancer” Cancers (Basel). 2019 Sep. 22; 11(10):1415. doi: 10.3390/cancers11101415. PMID: 31546746; PMCID: PMC6827145.
Sanchis-Gomar F. “Sestrins: novel antioxidant and AMPK-modulating functions regulated by exercise?” J Cell Physiol. 2013 August; 228(8):1647-50. doi: 10.1002/jcp.24338. PMID: 23359071.
Segalés et al. “Sestrin prevents atrophy of disused and aging muscles by integrating anabolic and catabolic signals” Nat Commun. 2020 Jan. 13; 11(1):189. doi: 10.1038/s41467-019-13832-9. PMID: 31929511; PMCID: PMC6955241.
Shen et al. “Suppression of Sestrins in aging and osteoarthritic cartilage: dysfunction of an important stress defense mechanism” Osteoarthritis Cartilage. 2017 February; 25(2):287-296. doi: 10.1016/j.joca.2016.09.017. Epub 2016 Sep. 29. PMID: 27693501; PMCID: PMC5258682.
Shi et al. “Sestrins: A New Kid for Stroke Treatment?” Curr Drug Deliv. 2017 Scp 6; 14(6):797-806. doi: 10.2174/1567201814666161111125249. PMID: 27834147.
Shi et al. “Sestrin2, as a negative feedback regulator of mTOR, provides neuroprotection by activation AMPK phosphorylation in neonatal hypoxic-ischemic encephalopathy in rat pups” J Cereb Blood Flow Metab. 2017 April; 37(4): 1447-1460. doi: 10.1177/0271678X16656201. Epub 2016 Jan. 1. PMID: 27381825; PMCID: PMC5453464.
Sun et al. “Sestrin proteins in cardiovascular disease” Clin Chim Acta. 2020 September; 508:43-46. doi: 10.1016/j.cca.2020.05.013. Epub 2020 May 11. PMID: 32407780.
Sun et al. “Aerobic exercise inhibits inflammatory response in atherosclerosis via Sestrin1 protein” Exp Gerontol. 2021 November; 155: 111581. doi: 10.1016/j.exger.2021.111581. Epub 2021 Oct. 8. PMID: 34634412.
Sun et al. “Sestrin2 overexpression attenuates osteoarthritis pain via induction of AMPK/PGC-1a-mediated mitochondrial biogenesis and suppression of neuroinflammation” Brain Behav Immun. 2022 May; 102:53-70. doi: 10.1016/j.bbi.2022.02.015. Epub 2022 Feb. 10. PMID: 35151829.
Sundararajan et al. “Decreased Sestrin levels in patients with type 2 diabetes and dyslipidemia and their association with the severity of atherogenic index” J Endocrinol Invest. 2021 July; 44(7):1395-1405. doi: 10.1007/s40618-020-01429-9. Epub 2020 Oct. 13. PMID: 33048307.
Tu et al. “Sestrin-Mediated Inhibition of Stress-Induced Intervertebral Disc Degradation Through the Enhancement of Autophagy,” Cell Physiol Biochem. 2018; 45(5): 1940-1954. doi: 10.1159/000487970. Epub 2018 Mar. 2. Erratum in: Cell Physiol Biochem. 2020; 54(5): 1088-1089. PMID: 29518770.
Wang et al. “Exercise improves glucose uptake in murine myotubes through the AMPKa2-mediated induction of Sestrins” Biochim Biophys Acta Mol Basis Dis. 2018 October; 1864(10):3368-3377. doi: 10.1016/j.bbadis.2018.07.023. Epub 2018 Jul. 23. PMID: 30048751.
Wang et al. “Cardiac SIRTI ameliorates doxorubicin-induced cardiotoxicity by targeting sestrin 2” Redox Biol. 2022 June; 52: 102310. doi: 10.1016/j.redox.2022.102310. Epub 2022 Apr. 6. PMID: 35452917; PMCID: PMC9043985.
Yi et al. “Upregulation of sestrin-2 expression protects against endothelial toxicity of angiotensin II” Cell Biol Toxicol. 2014 June; 30(3):147-56. doi: 10.1007/s10565-014-9276-3. Epub 2014 May 18. PMID: 24838122; PMCID: PMC4040185.
Zeng et al. “Sestrins are differentially expressed with age in the skeletal muscle of men: A cross-sectional analysis” Exp Gerontol. 2018 September; 110:23-34. doi: 10.1016/j.exger.2018.05.006. Epub 2018 May 8. PMID: 29751091.
Zhang et al. “Loss of sestrin 2 potentiates the early onset of age-related sensory cell degeneration in the cochlea” Neuroscience. 2017 Oct. 11; 361:179-191. doi: 10.1016/j.neuroscience.2017.08.015. Epub 2017 Aug. 15. PMID: 28818524; PMCID: PMC5605466.
Zhou et al. “Upregulation of sestrin-2 expression via P53 protects against 1-methyl-4-phenylpyridinium (MPP+) neurotoxicity” J Mol Neurosci. 2013 November; 51(3):967-75. doi: 10.1007/s12031-013-0081-x. PMID: 23959424.
U.S. Pat. No. 8,442,653 to Gill (May 14, 2013) for “Brain Electrode.”
U.S. Patent Application Publication US 2020-0289826-A1 to Leonhardt et al. (Sep. 17, 2020) for “Klotho Modulation.”
U.S. Patent Application Publication US 2021-0402184-A1 to Leonhardt et al. (Dec. 31, 2021) for “Klotho Modulation.”
U.S. Pat. No. 10,960,206 to Leonhardt et al. (Mar. 30, 2021) for “Bioelectric Stimulator.”

What is claimed is:

1. A bioelectric stimulator comprising an electric signal generator and electrode(s), which electric signal generator is programmed to produce at least one bioelectric signal that stimulates target tissue comprising living cells so as to modulate expression and/or release of a sestrin by living cells of the target tissue,
wherein the bioelectric signal comprises a signal having a biphasic constant current at a frequency of, within 15%, 10 Hz to 20 Hz, with a pulse width of, within 15%, 300 to 400 microseconds (μsec).

2. The bioelectric stimulator of claim 1, which bioelectric signal upregulates expression of sestrin(s) by the living cells.

3. The bioelectric stimulator of claim 2, wherein the bioelectric signal has a current of from 1 mA to 5 mA as may be measured at the level of the stimulated living cells of the target tissue.

4. The bioelectric stimulator of claim 3, wherein the bioelectric signal has a current of from 3 mA to 5 mA as may be measured at the level of the stimulated living cells of the target tissue.

5. The bioelectric stimulator of claim 1, wherein the bioelectric signal comprises a signal having a biphasic constant current at a frequency of, within 15%, 10 Hz, with a pulse width of, within 15%, 300 μsec.

6. The bioelectric stimulator of claim 1, wherein the bioelectric signal comprises a signal having a biphasic constant current at a frequency of, within 15%, 20 Hz, with a pulse width of, within 15%, 400 μsec.

7. A method of using the bioelectric stimulator of claim 1 to stimulate target tissue comprising living cells of a subject to modulate the expression of at least one sestrin by the living cells, the method comprising:
administering the bioelectric signal to the living cells via the electrode(s) for from about 5 minutes to about an hour, so as to upregulate the expression of at least one sestrin by the living cells.

8. The method according to claim 7, wherein the subject is suffering from heart disease, cancer, muscle atrophy, skin disorder(s), hair loss, Alzheimer's disease, Parkinson's disease, dementia, cognitive function, pre-mature aging related disorder, senescence, aging, muscle atrophy, and/or inflammation.

9. A method of using the bioelectric stimulator of claim 1 to stimulate target tissue comprising living cells of a subject to modulate the expression of at least one sestrin by the living cells, the method comprising:
administering the bioelectric signal to the living cells via the electrode(s) for greater than 90 minutes so as to downregulate the expression of the at least one sestrin by the living cells.

10. A method of modulating expression and/or release of at least one sestrin by a living cell, the method comprising:
applying to the living cell a bioelectric signal that comprises a signal having a biphasic constant current at a frequency of, within 15%, 10 Hz to 20 Hz, with a pulse width of, within 15%, 300 to 400 microseconds (μsec).

11. The method according to claim 10, wherein the bioelectric signal has a current of from 3 mA to 5 mA as may be measured at the level of the stimulated living cells of the target tissue.

12. The method according to claim 10, wherein the bioelectric signal comprises a signal having a biphasic constant current at a frequency of, within 15%, 10 Hz, with a pulse width of, within 15%, 300 μsec.

13. The method according to claim 10, wherein the bioelectric signal comprises a signal having a biphasic constant current at a frequency of, within 15%, 20 Hz, with a pulse width of, within 15%, 400 μsec.

14. The method according to claim 10, wherein the living cell is comprised within a subject.

15. The method according to claim 14, wherein the subject is suffering from heart disease, cancer, muscle atrophy, skin disorder(s), hair loss, Alzheimer's disease, Parkinson's disease, dementia, cognitive function, pre-mature aging related disorder, senescence, aging, muscle atrophy, and/or inflammation.

16. The method according to claim 11, wherein the bioelectric signal comprises a signal having a biphasic constant current at a frequency of, within 15%, 10 Hz, with a pulse width of, within 15%, 300 μsec.

17. The method according to claim 11, wherein the bioelectric signal comprises a signal having a biphasic constant current at a frequency of, within 15%, 20 Hz, with a pulse width of, within 15%, 400 μsec.

18. The method according to claim 11, wherein the living cell is comprised within a subject.

19. The method according to claim 18, wherein the subject is suffering from heart disease, cancer, muscle atrophy, skin disorder(s), hair loss, Alzheimer's disease, Parkinson's disease, dementia, cognitive function, pre-mature aging related disorder, senescence, aging, muscle atrophy, and/or inflammation.
